(19) United States
(12) Patent Application Publication
(10) Pub. No.: US 2023/0210600 A1
(43) Pub. Date: Jul. 6, 2023

(54) Microcatheter path generation method, shaping method of mandrel, computer equipment, readable storage medium and program product

(71) Applicant: Hangzhou ArteryFlow Technology Co., Ltd., Hangzhou (CN)

(72) Inventors: Yejie SHAN, Hangzhou (CN); Xiaochang LENG, Hangzhou (CN); Jianping XIANG, Hangzhou (CN)

(21) Appl. No.: 18/091,172

(22) Filed: Dec. 29, 2022

(30) Foreign Application Priority Data
Dec. 31, 2021 (CN) 2021116638807

(51) Int. Cl. A61B 34/10

(52) U.S. Cl. CPC A61B 34/10; A61B 2034/105; A61B 2034/107

(57) ABSTRACT

This application relates to an improved method for generating microcatheter paths, a method for shaping a mandrel, a computer device, a readable storage medium, and a program product. The method for generating the microcatheter path includes obtaining a cerebral vascular model, generating a centerline, determining the proximal starting point of the centerline, and successively generating a plurality of unit segments from the proximal starting point towards the distal aneurysm. Each unit segment includes a series of connected straight segments and curved segments, and the unit segments are successively connected to form at least a part of the microcatheter path. The generation method includes obtaining the starting point and slope of the straight segment, extending continuously from the starting point of the straight segment along the slope until contacting with the vascular wall, and obtaining the contact point

### CROSS-REFERENCE TO RELATED APPLICATION

This application claims priority from the Chinese patent application 2021116638807 filed Dec. 31, 2021, the content of which is incorporated herein in the entirety by reference.

### TECHNICAL FIELD

The present application relates to the technical field of medical devices, in particular to an improved microcatheter path generation method, a shaping method of mandrel, computer equipment, readable storage media and program products.

### BACKGROUND

An intracranial aneurysm is an abnormal bulge on the wall of an intracranial artery, with a prevalence of about 2%. Currently, the most common treatment for aneurysms is coil embolization or stent-assisted coil embolization. During this operation, the successful positioning and stability of the tip of the microcatheter play a key role in the successful implementation of the operation.

To ensure proper positioning and stability, it is generally necessary to shape the tip of the microcatheter. The traditional shaping procedure is to first insert a metal mandrel into the tip of the microcatheter, then perform three-dimensional shaping on the mandrel according to the direction of the blood vessel and the angle between the blood vessel and the growth direction of the aneurysm, and then steam fumigate it, and finally cool it with saline to maintain its shape.

However, the traditional shaping technology strongly relies on the rich knowledge and experience of doctors, and there is a relatively steep learning curve for junior doctors. For patients, the high failure ratio of shaping increases the operation time and operation cost. Based on this, it is necessary to provide an improved microcatheter path generation method to solve the above technical problems.

### SUMMARY

The improved microcatheter path generation method of the present application includes:

Acquire the intracranial vascular model with aneurysm, generate the centerline from the proximal vessel entrance to the distal aneurysm, determine the proximal start point, distal end point, and middle demarcation point of the centerline, and a plurality of unit segments are sequentially generated from the proximal starting point toward the distal aneurysm, and the unit segments include a straight line segment and a curved segment connected in sequence, and each unit segment is connected in sequence to form at least a part of the microcatheter path;

The generation methods of each unit segment include:

Obtain the starting point of the straight line segment and the slope of the straight line segment, and continuously extend the straight line segment from the starting point of the straight line segment along the slope of the straight line segment until the straight line segment touches the inner wall of the blood vessel to obtain a contact point;

The contact point is the starting point of the curved segment, and the closest point with the closest distance to the contact point is selected on the centerline to obtain an offset vector directed from the closest point to the contact point;

According to the offset vector, the nearest point and its follow-up points on the centerline toward the distal end are offset in sequence until a critical point that does not exceed the vessel wall after the offset is found, and the critical point is the end point of the curved section.

Optionally, during the sequential generation of each of the unit segments, monitor the distance change between the end point of the straight line segment or the end point of the curved segment of the current unit segment and the intermediate boundary point, and stop generating the current unit segment if the distance increases. The position where the generation was stopped is used as the new demarcation point point;

Curvilinear interpolation is performed between the new demarcation point and the distal termination point to obtain at least a portion of the path of the microcatheter.

Optionally, in each unit segment, the method of generating the curved segment includes:

Obtain the starting point of the curved segment and the end point of the curved segment;

Restrict the points within the blood vessel wall through offsetting the points beyond the blood vessel wall, and connect them sequentially to generate the curved segments.

Optionally, the unit segment includes the first unit segment and other unit segments generated sequentially;

The starting point of the straight line segment of the first unit segment is the starting point of the proximal end, and the slope of the straight line segment of the first unit segment is specified manually, or automatically generated according to any two points on the centerline, or is the tangent at the starting point on the centerline.

Optionally, the starting point of the straight line segment of the other unit segment is the end point of the curved segment of the last generated unit segment;

The slope of the straight line segment of the other unit segments is obtained according to a vector pointing to the end point of the curved segment from a point before the end point of the curved segment.

Optionally, during or after the generating process of the first unit segment, it also includes bending the straight line segment of the first unit segment, specifically including:

According to the offset vector, the centerline between the proximal starting point and the closest point to the contact point is offset, and the offset distance of each point presents a linear distribution.

The present application also provides a shaping method of a mandrel, comprising:

Obtain the microcatheter path that the improved microcatheter path generation method as claimed in claim 1 obtains;

Perform calculation on the microcatheter path to obtain the shape of the mandrel.

The present application also provides a computer device, including a memory, a processor and a computer program stored on the memory, and the processor executes the computer program to realize the steps of the improved microcatheter path generation method described in the present application.

The present application also provides a computer-readable storage medium, on which a computer program is stored, and when the computer program is executed by a processor, the steps of the improved microcatheter path generation method described in the present application are implemented.

The present application also provides a computer program product, including computer instructions. When the computer instructions are executed by a processor, the steps of the improved microcatheter route generation method described in the present application are implemented.

This application has at least the following effects:

The improved microcatheter path generation method of this application uses the centerline offset technology to calculate and obtain several unit segments connected in sequence. The straight and curved segments included in each unit segment simulate the interaction between the microcatheter and the vessel wall during delivery. The obstacles that may be encountered during the intervention of the microcatheter path are predicted, and the microcatheter path obtained in this application can meet the clinical needs;

The shaping method of the mandrel of the present application uses the obtained microcatheter path to obtain the shape of the mandrel, which can be used to shape the microcatheter.

### BRIEF DESCRIPTION OF THE DRAWINGS

FIG. 1 is a schematic flow diagram of an improved microcatheter path generation method in an embodiment of the present application;

FIG. 2 is a schematic diagram of the microcatheter path obtained in an embodiment of the present application and the shape of the mandrel;

FIG. 3 is an internal structure diagram of a computer device in an embodiment of the present application.

### DETAILED DESCRIPTION OF THE EXAMPLE EMBODIMENTS

Traditional microcatheter shaping has a steep learning curve and a long period for doctors to improve their abilities. However, the results obtained by the existing microcatheter shaping methods are relatively uncertain, which causes inconvenience in clinical use.

In order to make the purpose, technical solution and advantages of the present application clearer, the present application will be further described in detail below in conjunction with the accompanying drawings and embodiments. It should be understood that the specific embodiments described here are only used to explain the present application, not to limit the present application.

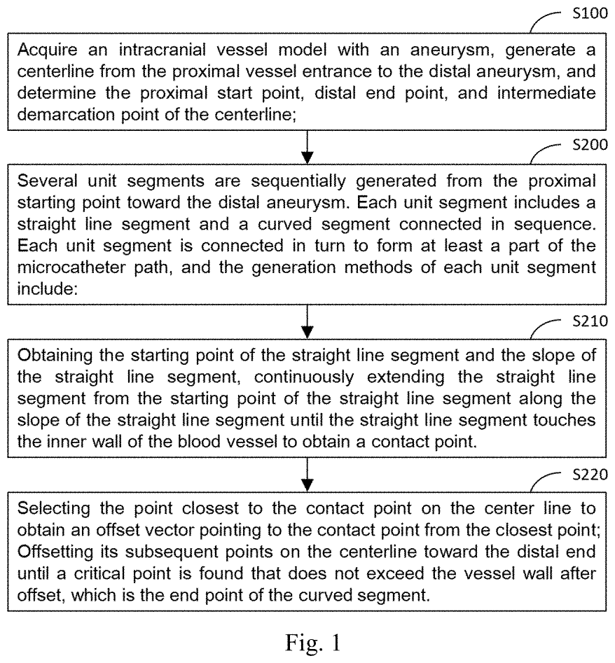

In order to solve the above technical problems, referring to FIG. 1, an improved microcatheter path generation method is provided in an embodiment of the present application, including:

Step S100, obtaining an intracranial vessel model with an aneurysm, generating a centerline from the proximal vessel entrance to the distal aneurysm, and determining the proximal start point, distal end point, and middle demarcation point of the centerline;

Step S100 specifically includes steps S110 to S130, wherein: Step S110, obtaining the medical image of the intracranial blood vessel, segmenting the medical image of the intracranial blood vessel by using the level set algorithm, and performing three-dimensional reconstruction on the image by using the marching cube algorithm, to obtain the intracranial blood vessel model.

Step S120, extracting the region with the aneurysm (that is, extracting the region of interest), and generating a center line from the proximal vessel entrance to the aneurysm. It can be understood that the path of the microcatheter to be generated is within the blood vessel, the distal end refers to the end relatively close to the aneurysm in the blood vessel with the aneurysm, and the proximal end refers to the end relatively far away from the aneurysm.

Step S130, selecting key points on the centerline, and the key points include the proximal start point, the distal end point, and the middle demarcation point. The middle demarcation point can be a point located at the neck of the aneurysm, or a point on the centerline of the vessel proximal to the aneurysm sac, which is selected by the user.

Step S200, generating several unit segments sequentially from the proximal starting point toward the distal aneurysm, each unit segment includes a straight line segment and a curved segment connected in sequence, and each unit segment is connected in sequence to form at least a part of the microcatheter path;

It can be understood that the straight section is the part of the microcatheter not constrained by the blood vessel wall, and the curved section is the part of the microcatheter that bends due to the constraint of the blood vessel wall. It is also possible that the curved part is that the microcatheter is not constrained by the vessel wall, but bends due to a change in direction (for example, in the case of branching vessels).

The generation method of each unit segment includes step S210 and step S220, wherein:

Step S210, obtaining the starting point of the straight line segment and the slope of the straight line segment, and continuously extending the straight line segment from the starting point of the straight line segment along the slope of the straight line segment until the straight line segment touches the inner wall of the blood vessel to obtain a contact point;

Step S220, the contact point is the starting point of the curved section, select the nearest point on the center line that is the closest to the contact point, and obtain the offset vector from the closest point to the contact point; Subsequent points towards the distal end are offset until a critical point is found that does not exceed the vessel wall after offset, which is the end point of the curved segment.

Further, in each unit segment, the method of generating the curved segment includes: obtaining the starting point and the end point of the curved segment; limiting the points beyond the vessel wall after offset within the vessel wall, and sequentially connecting them to generate the curved segment.

It can be understood that the centerline itself is composed of a series of discrete points, each discrete point has a distance from the contact point, and the discrete point on the centerline with the smallest distance from the contact point is selected as the closest point. The magnitude of the offset vector is the distance between the contact point and the closest point, and the direction is from the closest point to the contact point.

In the process of sequentially offsetting the nearest point and its subsequent points on the center line toward the distal end, calculate the distance D1 of each current point (that is, the point being offset) in contact with the vessel wall along the offset direction.

If the distance D1 is less than the magnitude of the offset vector, scale the magnitude of the offset vector to the distance, and use the scaled offset vector to offset the current point, that is, limit the point inside the vessel wall, points restrained inside the vessel wall are sequentially connected to form curved segments.

If the distance D1 is greater than or equal to the magnitude of the offset vector, that is, find the critical point that does not exceed the vessel wall after the offset, and the critical point is used as the end point of the curved segment and also as the starting point of the next straight segment.

Step S300, during the sequential generation of each unit segment, monitor the distance change between the end point of the straight segment or the end point of the curved segment of the current unit segment and the middle demarcation point, and stop generating the current unit segment if the distance increases (continue calculation and generate the unit segments if the distance monotonically decreases), use the position where the generation stops as a new demarcation point; perform curve interpolation between the new demarcation point and the distal termination point to obtain at least a part of the microcatheter path.

It can be understood that the microcatheter path includes two parts: the blood vessel segment from the proximal start point to the new demarcation point, and the extension segment between the new demarcation point and the distal end point. The blood vessel segment is formed by connecting several unit segments sequentially.

The curve interpolation may be, for example, using a Bezier curve or other curves to perform curve interpolation between the new demarcation point and the distal end point to obtain the extension segment. The curve interpolation can also be performed from the point on the unit segment before the new demarcation point to the end point, and the curve interpolation must ensure that the slope of the microcatheter path is continuous.

In one embodiment, before the final microcatheter path is generated, path smoothing is performed on the connected segments to obtain the final microcatheter path.

The improved microcatheter path generation method in this embodiment uses the centerline offset technology to calculate and obtain several unit segments connected in sequence. The straight and curved segments included in each unit segment simulate and predict the obstacles that may be encountered during the intervention of the microcatheter. The obtained microcatheter path can meet the clinical needs.

In step S200, the slope of the microcatheter path is continuous. The unit segment includes the first unit segment and other unit segments which are generated sequentially. For ease of understanding, this embodiment separately describes the generation process of the first unit segment and other unit segments.

For the first unit segment, the starting point of the straight line segment of the first unit segment is the proximal starting point, and the slope of the straight line segment of the first unit segment is specified manually, or is automatically generated according to any two points on the centerline, or is the tangent of the proximal starting point on the centerline.

It can be understood that the slope of the straight line segment of the first unit segment is the initial forward direction, and the slope of the straight line segment of the first unit segment can be obtained from the three-dimensional coordinates of any two points on the centerline, or can be specified interactively by the user. In this embodiment, the straight line segment of the first unit segment starts from the starting point at the proximal end of the centerline and extends along the initial forward direction to find a contact point with the vessel wall. The part of the path from the starting point at the proximal end to the contact point is a straight line segment, and the starting point from the contact point to the next straight line segment is a curved segment of the first unit segment.

During or after the generation process of the first unit segment, it also includes bending the straight line segment of the first unit segment, specifically including: deflecting the centerline between the proximal starting point and the nearest point according to the offset vector. The offset distance of each point presents a linear distribution. Specifically, the offset distance of the proximal starting point is zero, the offset distance of the nearest point is equal to the magnitude of the offset vector, and the offset distances of other discrete points that need to be offset gradually increase from the proximal end to the distal end, and present a linear distribution.

For other unit segments, the starting point of the straight line segment of other unit segments is the end point of the curved segment of last generated unit segment, and the starting point of the straight line segment of other unit segments can also be a discrete point before the end point of the curved segment of the last generated unit segment.

The slope of the straight line segment of other unit segments is obtained from the vector pointing to the end point of the curved segment from the point before the end point of the curved segment. It can be understood that the pointing manner of the slope of the straight line segment adopted in this embodiment ensures that the slope of each unit segment is continuous.

An embodiment of the present application also provides a shaping method of a mandrel. That is, step S400: perform calculation on the microcatheter path obtained in the above embodiments to obtain the shape of the mandrel.

The shape of the mandrel obtained in this example can be used to shape the microcatheter. Specifically, step 410 to step S430 are included, wherein:

Step S410, dividing the obtained microcatheter path into several tiny straight line segments;

Step S420, calculating the angle between any two adjacent straight line segments, and calculating the angle in combination with the inherent springback coefficient of the microcatheter;

Step S430, calculating the rotation axes of any two adjacent straight line segments, and calculating the rotation matrix of each straight line segment according to the angle and the rotation axis; using the rotation matrix to rotate all the straight line segments in turn to obtain the shape of the mandrel.

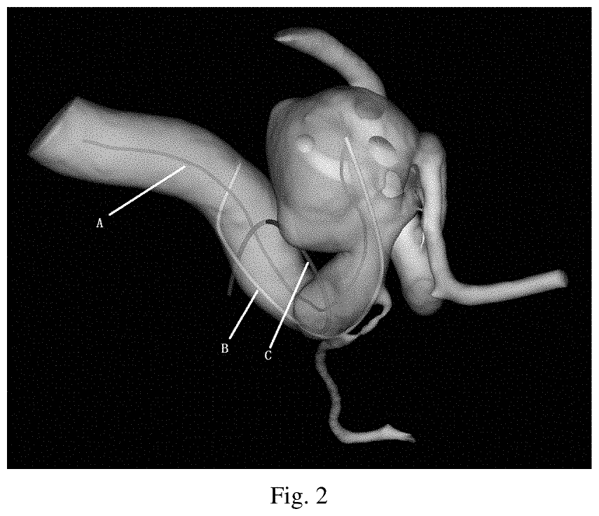

It can be understood that the shaping method of mandrel belongs to the application of the microcatheter paths obtained in the above embodiments. As shown in FIG. 2, the A line in FIG. 2 is the generated center line, the B line is the obtained microcatheter path, and the C line is the generated mandrel shape.

It should be understood that although steps S100 to S400 are numbered sequentially, these steps are not necessarily executed sequentially in numerical order. Unless otherwise specified herein, there is no strict order restriction on the execution of these steps, and these steps can be executed in other orders. Moreover, step S100, step S200, step S300 and step S400 may include multiple sub-steps or multiple stages, these sub-steps or stages are not necessarily executed at the same time, but may be executed at different times, The execution order of these sub-steps or stages is not necessarily performed sequentially, but may be executed alternately or alternately with at least a part of other steps or sub-steps or stages of other steps.

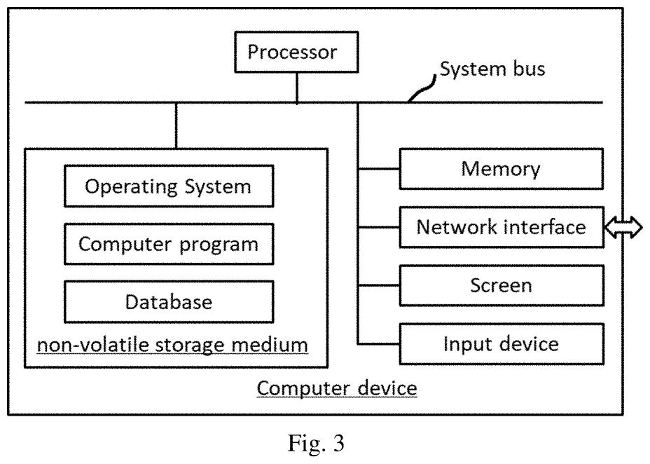

In one embodiment, a computer device is provided. The computer device may be a server, and its internal structure may be as shown in FIG. 3. The computer equipment includes a processor, a memory, a network interface and a database, a display screen and an input device connected by a system bus. Wherein, the processor of the computer device is used to provide calculation and control capabilities. The memory of the computer device includes a non-volatile storage medium and an internal memory. The non-volatile storage medium stores an operating system, computer programs and databases. The internal memory provides an environment for the operation of the operating system and computer programs in the non-volatile storage medium. The database of the computer equipment is used to store the data in each step of the improved microcatheter path generation method and the shaping method of the mandrel. The network interface of the computer device is used to communicate with an external terminal via a network connection. When the computer program is executed by the processor, an improved microcatheter path generation method and/or a shaping method of the mandrel can be realized.

The display screen of the computer device may be a liquid crystal display screen or an electronic ink display screen, which presents a three-dimensional visualization effect and can help users shape the microcatheter conveniently. The input device of the computer equipment may be a touch layer covered on the display screen, or a button, a trackball or a touch pad provided on the casing of the computer equipment, or an external keyboard, touch pad or mouse.

Users can visualize the vascular model, the final path of the microcatheter, and the shape of the mandrel in 3D on the computer device. Users can also measure the length and angle of the mandrel or microcatheter to facilitate the precise shaping of the microcatheter.

In one embodiment, a computer device is provided, including a memory and a processor, a computer program is stored in the memory, and the processor implements the following steps when executing the computer program:

Step S100, obtaining an intracranial vessel model with an aneurysm, generating a centerline from the proximal vessel entrance to the distal aneurysm, and determining the proximal start point, distal end point, and intermediate demarcation point of the centerline;

Step S200, generating several unit segments sequentially from the proximal starting point towards the distal aneurysm, the unit segments include a straight line segment and a curved segment connected in sequence, and each unit segment is connected in sequence to form at least a part of the microcatheter path;

The generation method of each unit segment includes step S210 and step S220, wherein:

Step S210, obtaining the starting point of the straight line segment and the slope of the straight line segment, and continuously extending the straight line segment from the starting point of the straight line segment along the slope of the straight line segment until the straight line segment touches the inner wall of the blood vessel to obtain a contact point;

Step S220, the contact point is the starting point of the curved section, select the nearest point on the centerline that is the closest to the contact point, and obtain the offset vector from the closest point to the contact point; Subsequent points towards the distal end are offset until a critical point is found that does not exceed the vessel wall after offset, which is the end point of the curved segment.

In one embodiment, when the processor executes the computer program, it also includes implementing the following steps:

Step S300, during the sequential generation of each unit segment, monitor the distance change between the end point of the straight segment or the end point of the curved segment of the current unit segment and the middle demarcation point, if the distance increases, stop generating the current unit segment, and use the position where the generation stopped as new demarcation point;

Curvilinear interpolation is performed between the new demarcation point and the distal termination point to obtain at least a portion of the microcatheter path.

In one embodiment, when the processor executes the computer program, it also includes implementing the following steps:

Step S400, perform calculation on the microcatheter paths obtained in the above embodiments to obtain the shape of the mandrel.

In one embodiment, a computer-readable storage medium is provided, on which a computer program is stored, and when the computer program is executed by a processor, the following steps are implemented:

Step S100, obtaining an intracranial vessel model with an aneurysm, generating a centerline from the proximal vessel entrance to the distal aneurysm, and determining the proximal start point, distal end point, and intermediate demarcation point of the centerline;

Step S200, generating several unit segments sequentially from the proximal starting point toward the distal aneurysm. Each unit segment includes a straight line segment and a curved segment connected in sequence, and each unit segment is connected in sequence to form at least a part of the microcatheter path;

The generation method of each unit segment includes step S210 and step S220, wherein:

Step S210, obtaining the starting point of the straight line segment and the slope of the straight line segment, and continuously extending the straight line segment from the starting point of the straight line segment along the slope of the straight line segment until the straight line segment touches the inner wall of the blood vessel to obtain a contact point;

Step S220, selecting the point on the center line which is the closest to the contact point, and obtaining the offset vector from the closest point to the contact point; Subsequent points towards the distal end are offset until a critical point is found that does not exceed the vessel wall after offset, which is the end point of the curved segment.

In one embodiment, when the computer program is executed by the processor, it also includes the following steps:

Step S300, during the sequential generation of each unit segment, monitor the distance change between the end point of the straight segment or the end point of the curved segment of the current unit segment and the middle demarcation point, if the distance increases, stop generating the current unit segment, and use the position where the generation stopped as new demarcation point;

Curvilinear interpolation is performed between the new demarcation point and the distal termination point to obtain at least a portion of the microcatheter path.

In one embodiment, when the computer program is executed by the processor, it also includes the following steps:

Step S400, performing calculation on the microcatheter path obtained in the above embodiments to obtain the shape of the mandrel.

In one embodiment, there is provided a computer program product comprising computer instructions which, when executed by a processor, implement the following steps:

Step S100, obtaining an intracranial vessel model with an aneurysm, generating a centerline from the proximal vessel entrance to the distal aneurysm, and determining the proximal start point, distal end point, and intermediate demarcation point of the centerline;

Step S200, generating several unit segments sequentially from the proximal starting point toward the distal aneurysm, each unit segment includes a straight line segment and a curved segment connected in sequence, and each unit segment is connected in sequence to form at least a part of the microcatheter path;

The generation method of each unit segment includes step S210 and step S220, wherein:

Step S210, obtaining the starting point of the straight line segment and the slope of the straight line segment, and continuously extending the straight line segment from the starting point of the straight line segment along the slope of the straight line segment until the straight line segment touches the inner wall of the blood vessel to obtain a contact point;

Step S220, the contact point is the starting point of the curved section, select the point on the centerline that is the closest to the contact point, and obtain the offset vector from the closest point to the contact point; Subsequent points towards the distal end are offset until a critical point is found that does not exceed the vessel wall after offset, which is the end point of the curved segment.

In one embodiment, when the computer instructions are executed by the processor, the following steps are implemented:

Step S300, during the sequential generation of each unit segment, monitor the distance change between the end point of the straight segment or the end point of the curved segment of the current unit segment and the middle demarcation point, if the distance increases, stop generating the current unit segment, and use the position where the generation stopped as new demarcation point;

Curvilinear interpolation is performed between the new demarcation point and the distal termination point to obtain at least a portion of the microcatheter path.

In one embodiment, when the computer instructions are executed by the processor, the following steps are implemented:

Step S400, perform calculation on the microcatheter paths obtained in the above embodiments to obtain the shape of the mandrel.

In this embodiment, the computer program product includes a program code part, which is used to execute the steps of microcatheter path generation method and/or the mandrel shaping method in each embodiment of the present application when the computer program product is executed by one or more computing devices. A computer program product can be stored on a computer readable recording medium. The computer program product may also be provided for download via a data network (e.g., via the RAN, via the Internet and/or via the RBS). Alternatively or additionally, the method may be coded in a field programmable gate array (FPGA) and/or an application specific integrated circuit (ASIC), or the functionality may be provided for download by means of a hardware description language.

Those of ordinary skill in the field can understand that all or part of the processes in the methods of the above embodiments can be implemented through computer programs to instruct related hardware, and the computer programs can be stored in a non-volatile computer-readable memory in the medium, when the computer program is executed, it may include the processes of the embodiments of the above-mentioned methods. Wherein, any references to memory, storage, database or other media used in the various embodiments provided in the present application may include non-volatile and/or volatile memory. Nonvolatile memory can include read only memory (ROM), programmable ROM (PROM), electrically programmable ROM (EPROM), electrically erasable programmable ROM (EEPROM), or flash memory. Volatile memory can include random access memory (RAM) or external cache memory. By way of illustration and not limitation, RAM is available in many forms such as Static RAM (SRAM), Dynamic RAM (DRAM), Synchronous DRAM (SDRAM), Double Data Rate SDRAM (DDRSDRAM), Enhanced SDRAM (ESDRAM), Synchronous Chain Synchlink DRAM (SLDRAM), memory bus (Rambus) direct RAM (RDRAM), direct memory bus dynamic RAM (DRDRAM), and memory bus dynamic RAM (RDRAM), etc.

Each embodiment of the present application can quickly and accurately obtain a microcatheter shaping solution that meets clinical needs. By performing simple interactive operations on the automatic, real-time, and accurate microcatheter shaping auxiliary software, the shape of mandrel can be obtained. This method flattens the learning curve of microcatheter shaping, lowers the technical threshold of microcatheter shaping, reduces the difficulty of surgery, and improves the hospital's ability to treat patients with aneurysms; it shortens the operation time, reduces the operation cost, and reduces the pain of patients. It has significant clinical application value and broad market prospect.

The technical features of the above embodiments can be combined arbitrarily. To make the description concise, all possible combinations of the technical features in the above embodiments are not described. However, as long as there is no contradiction in the combination of these technical features, it is considered to be within the range described in this specification. When the technical features in different embodiments are embodied in the same drawing, it can be considered that the drawing also discloses the combination examples of the various embodiments involved.

The above-mentioned embodiments only represent several implementation modes of the present application, and the description thereof is relatively specific and detailed, but it should not be construed as limiting the scope of the patent for the invention. It should be noted that those skilled in the field can make several modifications and improvements without departing from the concept of the present application, and these all belong to the protection scope of the present application. Therefore, the scope of protection of the patent application should be based on the appended claims.

1. A improved microcatheter path generation method, comprising:
acquiring the intracranial vascular model with aneurysm, generating the centerline from the proximal vessel entrance to the distal aneurysm, and determining the proximal start point, distal end point, and middle demarcation point of the centerline, wherein a plurality of unit segments are sequentially generated from the proximal starting point toward the distal aneurysm, and the unit segments include a straight line segment and a curved segment connected in sequence, and each unit segment is connected in sequence to form at least a part of the microcatheter path;
the generation methods of each unit segment include:
obtaining the starting point of the straight line segment and the slope of the straight line segment, and continuously extending the straight line segment from the starting point of the straight line segment along the slope of the straight line segment until the straight line segment touches the inner wall of the blood vessel to obtain a contact point; wherein
the contact point is the starting point of the curved segment, and the closest point with the closest distance to the contact point is selected on the centerline to obtain an offset vector directed from the closest point to the contact point; and
according to the offset vector, the nearest point and its follow-up points on the centerline toward the distal end are offset in sequence until a critical point that does not exceed the vessel wall after the offset is found, and the critical point is the end point of the curved section.

2. The improved microcatheter path generation method according to claim 1, comprising in the process that each described unit segment is generated successively, monitoring the distance of straight line end point or curved section end point of current unit segment and described middle demarcation point;
if the distance increases, stopping generating the current unit segment, and using the position where the generation stops at as the new demarcation point; wherein
curvilinear interpolation is performed between the new demarcation point and the distal end point to obtain at least a portion of the path of the microcatheter.

3. The improved microcatheter path generation method according to claim 1, wherein each unit segment, the generation approach of described curved segment comprises:
obtaining the starting point of the curved segment and the end point of the curved segment; and
restricting the points within the blood vessel wall through offsetting the points beyond the blood vessel wall, and connecting them sequentially to generate the curved segments.

4. The improved microcatheter path generation method according to claim 1, wherein described unit segment comprises the first unit segment and other unit segments which are generate successively; and
the starting point of the straight line segment of the first unit segment is the starting point of the proximal end, and the slope of the straight line segment of the first unit segment is specified manually, or automatically generated according to any two points on the centerline, or is the tangent at the starting point on the centerline.

5. The improved microcatheter path generating method according to claim 4, wherein the starting point of straight line segment of described other unit segments is the end point of the curved segment of last unit segment; and
the slope of the straight line segment of the other unit segments is obtained according to a vector pointing to the end point of the curved segment from a point before the end point of the curved segment.

6. The improved microcatheter path generating method according to claim 4, wherein in the generating process of the first unit segment or after the generating process, further comprising performing bending treatment on the straight line segment of the first unit segment, comprising:
according to the offset vector, the centerline between the proximal starting point and the closest point to the contact point is offset, and the offset distance of each point presents a linear distribution.

7. The shaping method of mandrel, comprises:
obtaining the microcatheter path that the improved microcatheter path generation method as claimed in claim 1 obtains; and
performing calculation on the microcatheter path to obtain the shape of the mandrel.

8. A computer equipment, comprising a memory, a processor and a computer program stored on the memory, wherein the processor executes the computer program to realize the steps of improved microcatheter path generation method according to claim 1.

9. A computer-readable storage medium on which a computer program is stored, wherein the computer program is executed by a processor to realize the steps of improved microcatheter path generation method according to claim 1.

10. A computer program product, comprising computer instructions, wherein the computer instructions are executed by a processor to realize the steps of improved microcatheter path generation method according to claim 1.

11. The computer equipment of claim 8, wherein the process that each described unit segment is generated successively, monitoring the distance of straight line end point or curved section end point of current unit segment and described middle demarcation point;
if the distance increases, stopping generating the current unit segment, and using the position where the generation stops at as the new demarcation point; wherein
curvilinear interpolation is performed between the new demarcation point and the distal end point to obtain at least the portion of the path of the microcatheter.

12. The computer equipment of claim 8, wherein each unit segment, the generation approach of described curved segment comprises:
obtaining the starting point of the curved segment and the end point of the curved segment; and
restricting the points within the blood vessel wall through offsetting the points beyond the blood vessel wall, and connecting them sequentially to generate the curved segments.

13. The computer equipment of claim 8, wherein described unit segment comprises the first unit segment and other unit segments which are generate successively; and
the starting point of the straight line segment of the first unit segment is the starting point of the proximal end, and the slope of the straight line segment of the first unit segment is specified manually, or automatically generated according to any two points on the centerline, or is the tangent at the starting point on the centerline.

14. The computer equipment of claim 13, wherein the starting point of straight line segment of described other unit segments is the end point of the curved segment of last unit segment; and
the slope of the straight line segment of the other unit segments is obtained according to a vector pointing to the end point of the curved segment from a point before the end point of the curved segment.

15. The computer equipment of claim 13, wherein in the generating process of the first unit segment or after the generating process, further comprising performing bending treatment on the straight line segment of the first unit segment, comprising:
according to the offset vector, the centerline between the proximal starting point and the closest point to the contact point is offset, and the offset distance of each point presents a linear distribution.

16. The computer-readable storage medium of claim 9, wherein the process that each described unit segment is generated successively, monitoring the distance of straight line end point or curved section end point of current unit segment and described middle demarcation point;
if the distance increases, stopping generating the current unit segment, and using the position where the generation stops at as the new demarcation point; wherein
curvilinear interpolation is performed between the new demarcation point and the distal end point to obtain at least the portion of the path of the microcatheter.

17. The computer-readable storage medium of claim 9, wherein each unit segment, the generation approach of described curved segment comprises:
obtaining the starting point of the curved segment and the end point of the curved segment; and
restricting the points within the blood vessel wall through offsetting the points beyond the blood vessel wall, and connecting them sequentially to generate the curved segments.

18. The computer-readable storage medium of claim 9, wherein described unit segment comprises the first unit segment and other unit segments which are generate successively; and
the starting point of the straight line segment of the first unit segment is the starting point of the proximal end, and the slope of the straight line segment of the first unit segment is specified manually, or automatically generated according to any two points on the centerline, or is the tangent at the starting point on the centerline.

19. The computer-readable storage medium of claim 18, wherein the starting point of straight line segment of described other unit segments is the end point of the curved segment of last unit segment; and
the slope of the straight line segment of the other unit segments is obtained according to a vector pointing to the end point of the curved segment from a point before the end point of the curved segment.

20. The computer-readable storage medium of claim 18, wherein in the generating process of the first unit segment or after the generating process, further comprising performing bending treatment on the straight line segment of the first unit segment, comprising:
according to the offset vector, the centerline between the proximal starting point and the closest point to the contact point is offset, and the offset distance of each point presents a linear distribution.
